(19) United States
(12) Patent Application Publication
(10) Pub. No.: US 2016/0242951 A1
(43) Pub. Date: Aug. 25, 2016

(54) Tooth Attachment

(71) Applicants: Bianca Berk, Marburg (DE); Joerg Koehler, Munchen (DE); Gotthard Sauer-Sperling, Starnberg-Perchting (DE)

(72) Inventors: Bianca Berk, Marburg (DE); Joerg Koehler, Munchen (DE); Gotthard Sauer-Sperling, Starnberg-Perchting (DE)

(21) Appl. No.: 14/128,063

(22) PCT Filed: Jun. 21, 2012
(86) PCT No.: PCT/EP2012/062015

§ 371 Date: Jul. 7, 2014

(30) Foreign Application Priority Data
Jun. 21, 2011 (DE) 202011102064.2

(51) Int. Cl. A61F 5/56; A61B 5/00

(52) U.S. Cl. CPC A61F 5/566; A61B 5/4557; A61B 5/4836; A61B 5/7405; A61B 5/7455; A61B 5/682; A61B 2562/0247; A61F 2005/563; A61B 2562/12

(57) ABSTRACT

The invention relates to a dental splint, comprising a base unit, in particular having dental splint plates irreversibly connected to each other, at least power supply unit, at least one signal generator, and at least one pressure sensor, which is operatively connected to the power supply unit and the signal generator, wherein the pressure sensor is designed in such a way that the pressure sensor detects both occlusion forces and protrusion forces acting on the pressure sensor.

The present invention concerns bite trays, among other things, as well as methods for making and methods for using such bite trays.

In the sense of the present invention, by the term bite tray is meant quite generally a device which is suited to suppressing or preventing the pressing and rubbing together of teeth or chewing surfaces of teeth, especially during sleep, and to eliminate or at least alleviate the consequences of so called grinding.

Patients who press their teeth together are known as grinders. The unconscious rubbing and pressing movements of the upper and lower jaw relative to each other are also known as bruxism (grinding of the teeth). Nocturnal bruxism (grinding of the teeth at night while sleeping) is a widespread ailment in industrialized countries. Most often the causes are of a psychological nature. Occupational, private and/or financial stress cause the patients to press their teeth together. A pressure of up to 80 kg/cm² may result. The grinding noises produced in this way are usually perceived by the affected persons and their family members as extremely annoying. A refreshing sleep is often not possible.

Bruxism can also lead to muscle, face, head and/or neck pain the next day. Furthermore, the tooth can become ground down in its substance and even the bone can be attacked by the permanent pressure bearing down on it.

The constant muscle contraction can lead to vessel constriction, which in turn can result in disturbed blood flow. This can cause tinnitus, or ringing in the ears. Many tinnitus patients are so-called grinders.

It is known how to use a tailor-made plastic tray for a grinder. However, this only prevents enamel abrasion. The above-mentioned symptoms are not prevented by such a tray.

Furthermore, it is know how to train patients. Thus, for example, it was proposed to stimulate the lips of patients by low electrical pulses during the grinding. The electrical pulse is not harmful, but it is painful, which does not meet with patient acceptance.

The problem of the present invention is to create a dental splint of improved action.

The problem is solved by a dental splint with the features of claim 1.

The dental splint comprises a base unit, especially one with dental splint plates irreversibly connected to each other. Preferably, these are an upper and a lower part of plastic dental splint plates. The dental splint furthermore comprises a power supply unit, which can be rechargeable. For example, it can be a customary battery or a customer storage cell.

Furthermore, the dental splint comprises at least one signal generator, for example, a vibration module with a vibration motor, and at least one pressure sensor, which is operatively connected to the power supply unit and the signal generator, the pressure sensor being configured such that it detects both occlusion forces and protrusion forces acting upon it.

Moreover, at least one electric circuit can be provided, which is arranged on a rigid or flexible circuit board, for example. The pressure sensor can be connected to the circuit and thereby enter into an operative connection with the signal generator and the power supply unit.

The pressure sensor or sensors can also be individually adapted to the particular user of the dental splint of the invention in dependence on a defined threshold value that is needed to generate the required signal by the signal generator. Furthermore, pressure sensors can also be used that relay any pressure signal at all, no matter how weak it is. In this case, the signal generator can be adjusted so that the desired signal which is supposed to prevent the pressing together of the chewing surfaces of the teeth or the sliding of the front teeth against each other is sent only at a predetermined pressure threshold value. This prevents the generating of a signal during swallowing, for example. Suitable pressure sensors can be provided accordingly for activation during different chewing pressures.

The pressure sensor is preferably at least partially embedded in a material that is compatible with or identical to the material of the base unit.

Preferably, the pressure sensor is at least partially embedded in a thermoplastic material and/or an elastomeric material, especially TPE material.

The dental splint can be, in particular, a plastic form that is molded so that it can be accommodated in the oral cavity in the manner of a denture.

For example, the dental splint can be individually adapted afterwards to the teeth of the user. In this way, the dental splint can constitute, in particular, an adaptable dental splint. Alternatively, the dental splint can be made in a mass production process, wherein the dental splints are produced in certain standardized sizes. In this case, the dental splint is not individually manufactured.

In a region facing the palate of the patient, the following electronic components can be installed: 1. vibration motor, 2. energy supply, such as a battery, storage cell, capacitor (chargeable, e.g., by means of induction or solar), 3. circuit board, outfitted with connections to 1. and 2., as well as the corresponding electronic components. A connection to a pressure sensor operating as a signal transmitter can also be provided.

The pressure sensor can be situated in the region between the dental ridges of the upper and lower jaw. If occlusion or protrusion forces occur, there is a flow of current, for example, between the power supply unit and signal generator and a signal is triggered, especially a vibration, of the entire dental splint. Thanks to the resulting vibration in the sensitive palate region and the resulting noise produced in the inner ear, by means of bone conduction for example, the patient can unconsciously learn to stop the grinding. Demonstrably better learning outcomes can be achieved by the use of two different senses, here, acoustical and vibrational, in a single device. Thanks to the stimuli, the unconscious grinding process can be intuitively interrupted. The muscles involved, especially the musculus masseter, muscular temporalis and musculus medialis, are directly involved close in time and space and relaxed by means of the stimuli. By this (bio)feedback mechanism, the body learns to prevent the undesirable action, i.e., the grinding. With regular use, the patient becomes trained by means of a negative stimulus, such as vibration, to suppress the undesirable grinding process.

The signal generating according to the invention occurs not only during the actual pressing and grinding, but also during the particularly often occurring sliding. Thus, an easily employed therapy option also exists for protrusion. This improves the treatment of nocturnal bruxism in particular.

Advantageous modifications of the invention will be found in the subclaims, the specification and the drawings.

According to one embodiment, the dental splint according to the invention can constitute a unit for the reversible fastening to at least one tooth, such as a molar, or a segment thereof.

In another embodiment, this dental splint constitutes a unit for the reversible fastening to at least two, three, four or more teeth, especially molars. The dental splint can constitute a unit for the reversible fastening to a cuspid or a cuspid and at least one neighboring tooth or a unit for the reversible fastening to a cuspid and at least one, especially at least two molars, and/or at least one cuspid and at least one, especially at least two, incisors.

Preferably, the dental splint according to the invention constitutes a unit for the reversible fastening to all or nearly all the teeth of the upper or lower jaw.

The dental splint can be fastened, e.g., to at least one, two, three, four or more premolars of the upper or lower jaw.

According to one embodiment, the pressure sensor comprises a contact device, which is adapted so that its electrical resistance changes in dependence on the strength of the occlusion forces and protrusion forces acting on it. The contact device can comprise, for example, at least one electrical switch. Thus, e.g., conducting wires can be provided, which only come into contact with each other due to the chewing pressure and in this way activate the signal generator. Once the chewing pressure relaxes, the circuit is broken once more, for example by an elastic and/or flexible material in which the wires are embedded. The contact device in the sense of the present invention can also be called a pressure switch or constitute such a switch. The contact device can also form part of the base unit or the unit itself. Furthermore, it is conceivable to provide two, three or more contact devices in the base unit. Often, however, it is enough to have only a single contact device to accomplish the desired effect of suppressing or preventing the tooth grinding with the dental splint according to the invention. It is also possible to arrange a single contact device in the region of the back grinding or cheek teeth (molars), in the region of the front grinding or cheek teeth (premolars), in the region of the cuspids (canines) and/or in the region of the front teeth (incisors) so that both occlusion and protrusion forces can be detected.

In one embodiment, the contact device is arranged along a common dental splint plate of the dental splint according to the invention or along nearly the entire extent of the dental splint plate. In this way, one can make sure that a signal is only sent when a predetermined pressure value is reached or exceeded by the opposite teeth making contact.

Alternatively, the pressure sensor can also comprise a hydraulic or pneumatic device, for example, one in the form of a pressurized sleeve. If the teeth press on the sleeve, the pressure increases. The pressure change can be measured, and after a certain minimum pressure a signal generator is triggered. In this way, information as to the strength of the acting occlusion and protrusion forces is relayed directly to the signal generator, for example, by a pressure change.

The forces needed to trigger a signal can be set by the design or the material of the contact device.

According to another embodiment, the contact device comprises at least one contact sleeve, which comprises in cross section at least four segments of conducting and nonconducting material alternating in the circumferential direction, especially a plastic material. The segments of the contact sleeve can essentially extend continuously in the longitudinal direction, for example. Alternatively, it is also conceivable that the segments extend only for a partial section of the contact sleeve. The contact sleeve can be hollow on the inside.

Alternatively or additionally, it can comprise at least one elastic and/or flexible material. The conducting and/or nonconducting material can be elastic and/or flexible in particular, so that it can be compressed when pressure is acting on it. In this way, a contact is produced between the conducting segments.

The contact sleeve is installed for example on one lead of the circuit board and thus functions as a switch. If the contact sleeve is compressed in a corresponding direction, a current flows in particular between battery and vibration motor and triggers a vibration of the entire dental splint. The contact sleeve or sensor sleeve can have a longitudinal resistance of around 50 to 180 kΩ/mm, especially around 80 kΩ/mm. The insulation resistance is, for example, greater than 20 kΩ. The sensor sleeve can have a length of up to 320 mm, for example. The trigger threshold for the signal generator is typically around 50 kΩ.

The segments in particular are configured as a ring circular in cross section with an inner diameter of around one millimeter and a thickness of around 0.4 mm. The sectors of conducting material extend in the cross section, for example, over around 100°, while the segments of nonconducting material extend over around 80°. The cross section of the contact sleeve is preferably round, while another geometrical shape such as an oval can also be used. In particular, the contact sleeve can be divided into more than four segments. For example, eight segments of alternating conducting and nonconducting material are also possible.

According to another embodiment, the contact sleeve can comprise several sections in the lengthwise dimension, wherein at least one first section, which in the installed condition of the dental splint is arranged especially behind and/or beneath the front incisors, is twisted relative to at least one second section, which in the installed condition of the dental splint is arranged especially at the cuspids, the premolars and/or the molars teeth. Thus, in particular, the same contact sleeve can be used for detecting of both occlusion forces and protrusion forces.

Alternatively, it is also possible to arrange several contact sleeves, twisted relative to each other, on the base unit. A twisting within a contact sleeve is not necessary in this case. Neither is this required, for example, when the contact sleeve has more than four, especially eight segments.

According to another embodiment, at least one part of the conducting material in the first section is arranged on either side of a frontal plane and/or at least one part of the conducting material in the second section is arranged on either side of a transverse plane. Both when the front teeth slide along a sagittal direction and when the molars are pressing for example in the longitudinal direction, a contact can be produced in this way. In particular, the contact sleeve can be placed in the region of the cuspids as well as the premolars and molars so that the electrically conducting layers are situated on either side of the transverse plane, and the nonconducting layers on either side of the frontal plane. In the classical grinding process, i.e., a pressing from above or below, the contact sleeve is compressed such that the two electrically conducting layers touch and a contact is produced. When the lower incisors slide forward against the upper incisors, here as well the contact sleeve is compressed, so that a contact arises, since the contact sleeve is installed in the region behind the front incisors twisted by roughly 90°, for example, so that the electrically conducting layers are situated on either side of the frontal plane and the nonconducting layers on either side of the transverse plane.

According to another embodiment, the pressure sensor comprises at least two sensors, which are oriented essentially perpendicular to each other.

According to another embodiment, the signal generator comprises a signal transmitter. This can be, e.g., a mechanical signal transmitter, an acoustical signal transmitter, an electrical signal transmitter and/or a thermal signal transmitter. With a combination of several signal transmitters, several senses of the patient are called upon during the grinding, which can improve the training

The mechanical signal transmitter can comprise a vibrator or a vibration module. Such vibration modules are known to the skilled person. For example, it can be a vibration module such as is used in mobile radio devices. The vibration module comprises in particular a vibration motor, which rotates, for example.

According to another embodiment, the dental splint comprises at least one storage, transmitting and/or evaluating unit. This is especially helpful for diagnostic purposes, in order to read out and evaluate the data gathered. For example, the circuit board can contain the storage unit, the transmitting unit and/or the evaluating unit.

The evaluating unit can be, e.g., at least one microprocessor, which is in operative connection with the pressure sensor and/or the signal generator and/or at least one transmitting unit. By using a storage and/or evaluating unit, the signals put out by the pressure sensor can be recorded and processed, e.g., in regard to the frequency of signal activation, the pressure intensity and the pressure duration. With the help of such a storage and/or evaluating unit, it is possible to track and document the healing process of the user of the dental splint. The data stored in the storage and/or evaluating unit, such as a microprocessor, can be read out via a separate unit, for example after the dental splint is removed from the mouth.

Alternatively or additionally, the transmitting unit can be operatively connected to the pressure sensor. Signals emitted by the pressure sensor can be relayed via this transmitting unit, e.g., through Bluetooth or infrared, to an external receiving unit and from there go to a storage and evaluating unit. Suitable transmitting and/or receiving units are familiar to the skilled person.

The data saved in the storage and/or evaluating unit can then be relayed via the transmitting unit to a corresponding receiving unit and presented for example on a monitor screen or optionally be printed out.

According to another embodiment, the signal generator and the power supply unit, and also optionally the storage, transmitting and/or evaluating unit, are an integral component of a module. The module in particular forms part of the base unit, or it is itself the base unit. The components are built into the module, in particular. This eases the handling of the dental splint.

According to another embodiment, a central unit which comprises the signal generator and the power supply unit, and also optionally the storage, transmitting and/or evaluating unit is at least partially enclosed by a separating film. Thanks to the separating film, the splint can be easily taken out. This ensures a proper and environmentally-friendly disposal of the electronic components. A replacement and/or a recycling of the central unit also becomes possible in this way.

According to another embodiment, the dental splint is formed entirely or partially from at least one thermoplastic polymer and/or at least one elastomer, such as TPE.

According to another embodiment, the power supply unit comprises a rechargeable energy store. The charging can take place by means of induction, for example. The energy store can be, e.g., a lithium ion battery as a secondary cell, i.e., a rechargeable storage cell. This can have a voltage rating of 3.7 Volts, for example. Up to 2000 triggerings per battery charge are conceivable. In particular, more than 500 charging cycles are provided. Alternatively, energy stores are also conceivable that are not rechargeable, for example, a primary cell of lithium-magnesium. Such primary cells can have a voltage rating of 3 Volts, for example. In this way, up to 18,000 triggerings are possible, for example. The primary cell can be permanently soldered into the circuit board, in particular.

With rechargeable energy stores the charging can be done, for example, through the power mains. The use of solar cells is also conceivable. It is also possible to integrate one or more solar module units into the dental splint of the invention and/or in a charging station, which is operatively connected to the power supply unit. For example, after using and cleaning it, the dental splint can be placed in sunlight and thereby charged. It is also conceivable to use a capacitor as the power supply unit.

The invention furthermore concerns a charging station for a dental splint according to the invention, which is designed as a stowage receptacle for the dental splint. The charging station is inductively coupled to the dental splint, so that in particular a rechargeable energy store of the dental splint can be charged. The charging station comprises in particular an inductive transmitter, as well as corresponding controls. The dental splint, on the other hand, comprises a receiving coil, which is preferably built into the vibration module.

The surface beneath the transmitter coil is preferably left open. During the installation, the transmitter coil is slanted upward, for example, in order to achieve the closest possible contact with the receiving coil. The transmitter coil of the charging station is designed in particular as a single-layer flat coil, e.g., on the top side of a circuit board.

The receiving coil of the dental splint can be multilayered and integrated in particular into the circuit board. For example, the coil can consist of around 50 windings and be part of a conductor structure of the circuit board, in which case the fabrication can be done in particular together with the rest of the circuit board. In particular, conducting tracks around 35 μm thick and 125 lam wide can be distributed over six layers of a multilayer. The transmitter coil of the charging station can also be designed as a circuit board coil, preferably as conductor tracks around 70 μm thick and 500 μm wide. The dimensions of the receiving coil in the vibration module can be around 25×20×0.8 mm, while the dimensions of the transmitter coil in the charging station can be around 30×28×1.5 mm.

There is some play present in regard to the charging position. Thus, in particular, the distance between the two coils during the charging can be variable and/or amount to around 8 mm. Depending on the distance, a certain lateral position tolerance can also be granted. Thus, the lateral position can vary by several millimeters, for example. In particular, a full charge of the secondary cell is made possible within 12 hours, within the tolerances.

The tolerance of the coils relative to each other allows for the fact that the dental splints can be adapted individually to the patients, so that the position of the receiver coils in the charging station is also not always the same for different dental splints.

The invention furthermore involves a method for making a dental splint according to the invention, comprising the providing of at least one base unit, especially one with dental splint plates that are irreversibly connected to each other, being milled or laser cut out from a monolithic block, darned from a fabric, cast from liquid plastic, produced in a polymerization, injection molding, deep drawing or laser sintering process, in the rapid prototyping or 3D printing process, CAD/CAM process or wax process and/or consisting of thermoplastic and/or elastomeric material, especially TPE material, comprising at least one power supply unit, especially one comprising a rechargeable energy store, at least one signal generator, and at least one pressure sensor, which is operatively connected to the power supply unit and the signal generator and optionally to a microprocessor, and the positioning of the pressure sensor so that it can detect both occlusion forces and protrusion forces acting on it. In particular, a contact sleeve can be installed twisted during the manufacturing process, so that occlusion and protrusion forces can be detected.

The dental splint in particular is a deep drawn splint, consisting of two layers of splint film with hermetically sealed central unit. This is made essentially by the same principle as normal bite trays. The dentist takes an impression, e.g., with alginate, Impregum, or another suitable material, of the upper and lower jaw and sends this to a dental laboratory. The dental laboratory makes from this impression a plaster model. In the next step, using a deep drawing device, a base film is deep drawn. The central unit is placed on this base splint, being embedded in a separating and/or isolating film, for example, making possible in particular a proper disposal. The contact sleeve is laid according to the tooth status, also depending on whether the pressing and grinding movements of the patient occur with or without lower jaw sideways movements and/or lower jaw forward movements.

According to another embodiment, in order to obtain the structure of the pressure sensor, especially the contact sleeve, a spacer such as a plastic thread is temporarily introduced into the pressure sensor, e.g., the contact sleeve, during the manufacturing of the dental splint. Especially in order to protect the contact sleeve before a compression, such as during a deep drawing process, a spacer can be introduced into it, especially centrally in the cross section, prior to the installation. The spacer can extend over the entire length of the contact sleeve.

For the sealing of the electronic parts and/or the pressure sensor, especially the contact sleeve, particularly after its placement on the base splint, a second film for example is deep drawn over the base splint, the electronic parts and/or the pressure sensor, especially the contact sleeve. After this, the spacer is once gain pulled out from the contact sleeve and the resulting hole in the film is sealed up again. In this way, one can ensure, in particular, that the contact sleeve is also hollow on the inside. This is desirable, since the sleeve can thereby change its shape and be compressed when subjected to pressure from the teeth.

A central unit, which comprises the signal generator and the power supply unit, as well as optionally the storage, transmitter and/or evaluating unit, can be enclosed at least partly by a separating film of a suitable material during the manufacturing of the dental splint.

Furthermore, the invention pertains to the using of a dental splint, especially one according to the invention, the detecting of the strength of acting occlusion forces and protrusion forces with the help of a pressure sensor, especially a contact device fashioned as a contact sleeve, whose electrical resistance changes in dependence on the strength of the occlusion forces and protrusion forces acting upon it, the direct or indirect relaying of the strength information to a signal generator, the generating of a signal by the signal generator when the strength exceeds a predetermined limit value, and the dropping out of the signal when the strength falls below the predetermined limit value, wherein the pressure sensor and the signal generator are energized from a power supply unit, comprising in particular a rechargeable energy store.

The present invention shall be described hereafter, merely in exemplary fashion, by means of advantageous embodiments making reference to the enclosed drawings. There are shown:

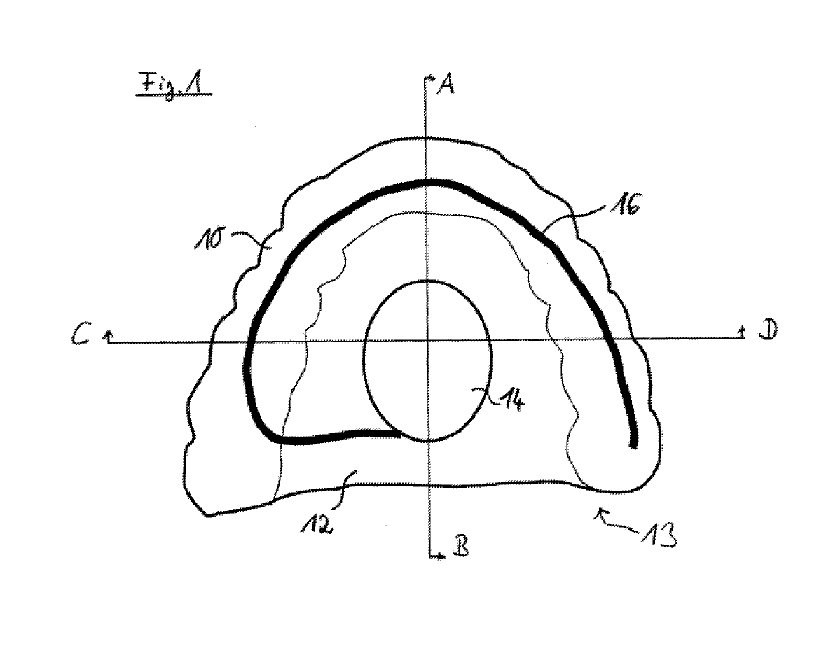

FIG. 1, a top view of one embodiment of a dental splint according to the invention,

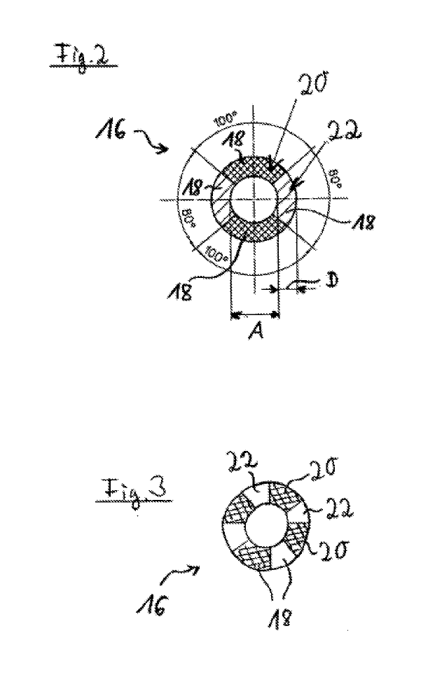

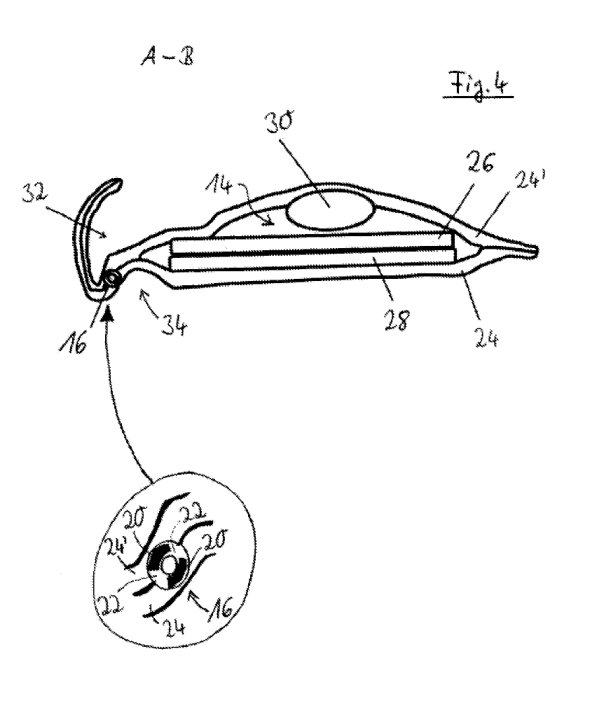

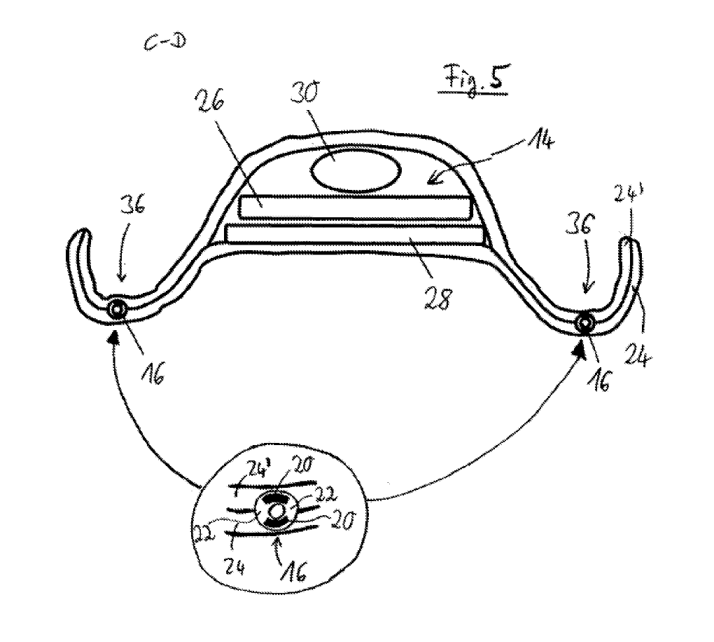

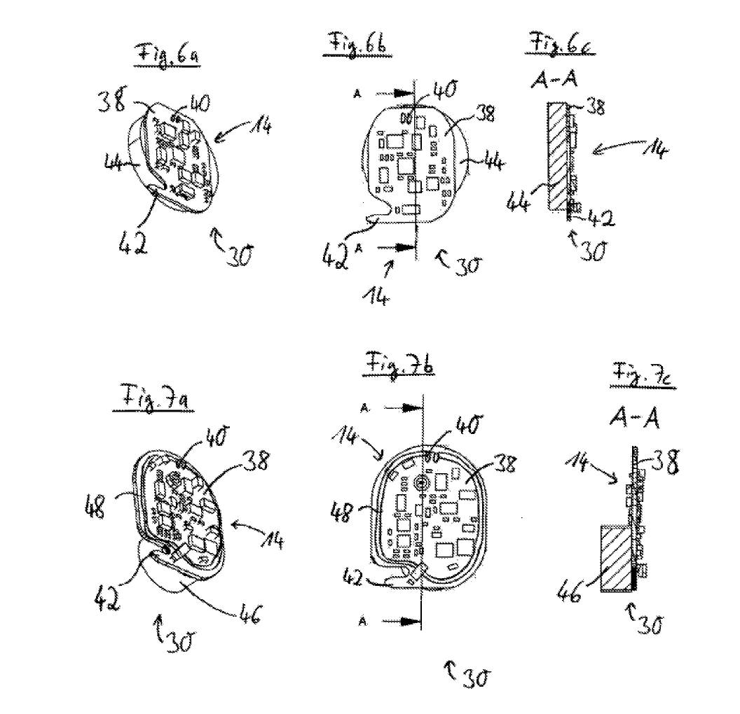

FIG. 2, a sectional view of one embodiment of a contact sleeve according to the invention,

FIG. 3, a sectional view of a second embodiment of a contact sleeve according to the invention,

FIG. 4, a sectional view according to FIG. 1 (A-B),

FIG. 5, a sectional view according to FIG. 1 (C-D),

FIG. 6a-6c, various views of a central unit with primary cell of a dental splint according to the invention,

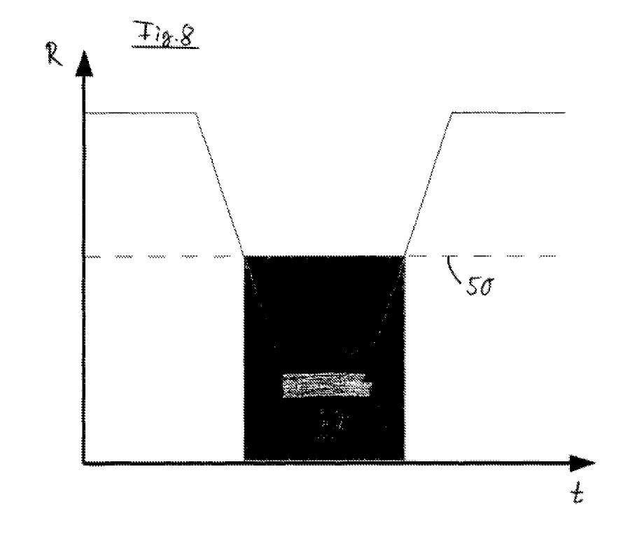

FIG. 7a-7c, various views of a central unit with secondary cell of a dental splint according to the invention,

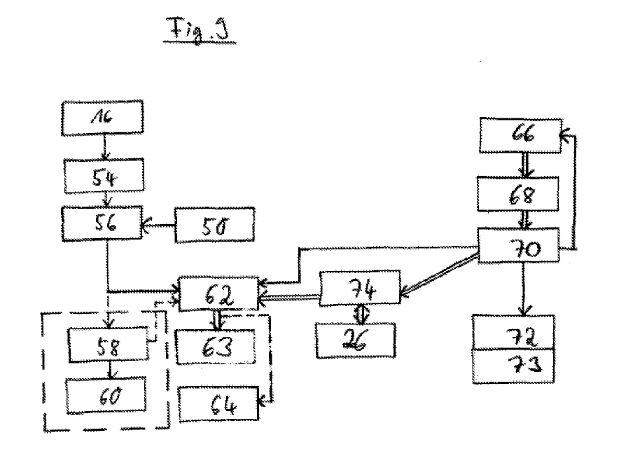

FIG. 8, a diagram showing a triggering threshold,

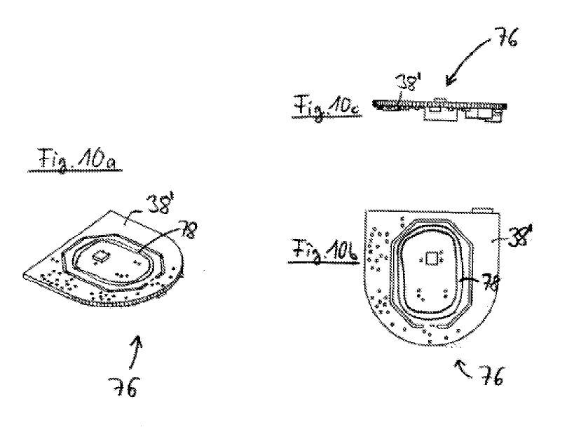

FIG. 9, a block diagram of a central unit of a dental splint according to the invention,

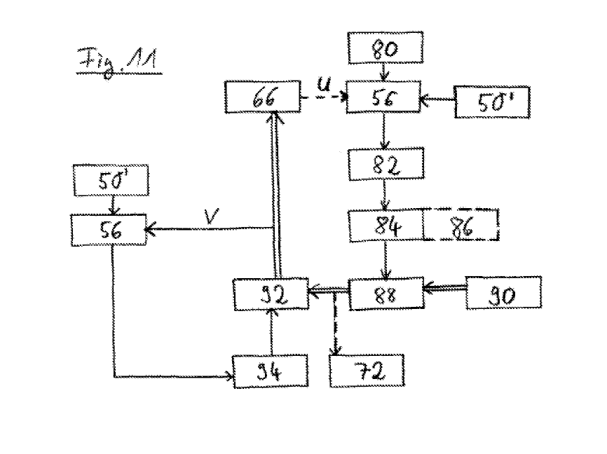

FIG. 10a-10c, various views of a charging station of a dental splint according to the invention, and

FIG. 11, a block diagram of a charging station of a dental splint according to the invention.

FIG. 1 shows a dental splint with a dental arch 10 and a palate piece 12. The dental arch 10 and the palate piece 12 together form a base unit 13. On the palate piece 12 is arranged a central unit 14, which can comprise for example an electronics with a vibration motor and a power supply unit.

In the region of the dental arch 10 there is arranged a pressure sensor as a contact device, being configured in this sample embodiment as a contact sleeve 16. The contact sleeve 16 is operatively connected to the central unit 14, in particular, electrically connected.

FIG. 2 shows a sectional view of the contact sleeve 16. This is divided into four compartments or segments 18 in cross section. The segments or sectors are each formed alternately of conducting material 20 and nonconducting material 22. The inner diameter A can be 1 mm, for example, while the thickness D can be around 0.4 mm. The nonconducting material 22 has an insulating action and comprises, in particular, a flexible material. Thus, this can be compressed with the occurring occlusion and protrusion forces, so that the two segments 18 with conducting material 20 can touch and thereby produce an electrical contact. This changes the electrical resistance of the contact sleeve 16, which is hollow on the inside, in particular.

FIG. 3 shows another embodiment of a contact sleeve 16. This comprises eight segments 18 and thus has four segments 18 of conducting material 20 and four of nonconducting material 22.

FIG. 4 shows a sectional view along line A-B of FIG. 1. Between a deep drawn film 24′, which is arranged on the palate side, and a deep drawn film 24 which is arranged on the tongue side, there is situated the central unit 14. This comprises a power supply unit 26 with an energy store, which can be fashioned as a battery or a storage cell, especially a rechargeable one. Furthermore, the central unit 14 can comprise electronics 28 and a signal generator, which in this sample embodiment is configured as a vibration module 30. The vibration module 30 comprises, e.g., a vibration motor, especially a rotary kind. Alternatively, it is also possible to omit the electronics 28 and hook up the vibration motor directly to the pressure sensor and the energy source.

Between the region of the upper incisors 32 and the region of the lower incisors 34 is arranged a contact sleeve 16. Depending on the individual tooth status, the contact sleeve can also be placed higher or lower than is shown in the drawing. A corresponding detail is also shown magnified in FIG. 4, together with the deep drawn films 24, 24′. The segments 18 with conducting material 20 are arranged at the side here, so that an electrical contact can be produced upon sideways movement of the incisors, i.e., a protrusion. The contact sleeve 16 corresponds to the embodiment of FIG. 2. Alternatively, it is also conceivable to use a contact sleeve 16, for example, according to FIG. 3. The contact sleeve 16 is positioned such that an electrical contact is produced between two conducting materials 20 in event of a protrusion.

FIG. 5 shows a sectional view along line C-D of FIG. 1. The contact sleeve 16 here is arranged on a region of the upper cheek teeth 36. The conducting material 20 is situated at the top and bottom in the contact sleeve 16 (see magnified view). Thus, an electrical contact is produced between the conducting materials 20 in event of occlusion forces.

The contact sleeve 16 is twisted in the region of the incisors 32, 34 (see FIG. 4) relative to the region of the upper cheek teeth 36 (see FIG. 5). If a contact sleeve 16 according to FIG. 3 is used, on the contrary, no twisting of the contact sleeve 16 is needed. Thus, in either case, it is possible to detect both occlusion and protrusion forces with the help of a single contact sleeve 16.

After a certain pressure, a signal is sent to the vibration module 30. Thereupon, a vibration is triggered, so that the patient stops grinding his teeth.

Various views of a sample embodiment of the central unit 14 are shown in FIGS. 6a, 6b and 6c. The central unit 14 comprises a circuit board 38 with a motor terminal 40. The motor is set off from the circuit plate 38 and therefore not depicted. Furthermore, the circuit board 38 comprises a terminal 42 for a contact sleeve 16. The terminal 42 is designed, e.g., as a partial margin copper plating of the circuit board 38. An optional gold plating prevents oxidation during storage and use. The circuit board 38 is arranged on a primary cell 44. It is also possible to connect the central unit 14 and the motor with the help of a common rigid-flex circuit board, in order to replace the connection wires at the motor.

The central unit 14 according to FIGS. 7a, 7b and 7c essentially corresponds to the central unit 14 of FIG. 6. The circuit board 38 in this embodiment, however, is arranged on a secondary cell 46. The central unit 14 thus has a rechargeable energy source. For the charging, the circuit board 38 can have a receiving coil 48, in particular.

FIG. 8 shows a diagram to represent the activating of a vibration. Here, the electrical resistance R is plotted against the time t. The broken line represents a trigger threshold 50 or limit value, while the gray area defines a vibration region 52. To activate the vibration, a contact sleeve 16 is used, whose change in resistance triggered by the pressing of the teeth is evaluated in the central unit 14. A comparator compares the sensor signal with a fixed threshold 50. Due to the pressing on the contact sleeve 16, an electrical contact is produced between the conducting materials 20, so that the resistance R decreases in the contact sleeve 16. If it falls below the trigger threshold 50, the vibration motor of the vibration module 30 is switched on and vibrates. Due to the vibration, the jaw ceases to exert pressure on the contact sleeve, so that the electrical resistance R increases in the contact sleeve 16. The vibration is again interrupted.

FIG. 9 shows a block diagram of a central unit 14. Single arrows here represent a transmitted signal, while double arrows symbolize the energy. A pressure sensor, such as a contact sleeve 16, at first detects the strength of the occlusion or protrusion. The signal is filtered in a filter 54 and goes to a comparator 56. Here, the signal is compared to a trigger threshold 50. Optionally, the signal is routed to a vibration control memory 58 and an optical data transmission unit 60. Energy arrives across a DC/DC converter 62 at a vibration motor 63. A display 64 can indicate whether the central unit 14 is active, for example. If a rechargeable battery or a storage cell is used, a transmitter 66 can furthermore be provided, which routes energy to a rectifier 68 and a charging controller 70. The charge status can be indicated by a charge indicator 72. A charge feedback 73 can be put out in this process. The charging controller 70 as well as a battery or a storage cell as the power supply unit 26 are connected to a charging and discharging protection 74. The DC/DC converter 62 is energized in this way.

The energy potential level is reduced to the operating potential of the vibration motor 63 by means of the dc voltage converter 62. When using a primary cell 44, the dc voltage converter 62 is replaced by a switch, since the operating potential levels will be similar in this case.

An independent charging and/or discharging protection 74 monitors the battery potential and/or the battery current and prevents further charging and/or discharging if necessary. This function is only completely provided when using a rechargeable battery, i.e., a secondary cell 46. With a primary cell 44, a safety fuse will be used, especially a self-resetting one.

The energy transmission during the charging occurs in particular by induction with the help of a coil integrated in the circuit board 38 (see FIG. 7a-c), which is tuned along with a capacitance to the transmission frequency. The rectified alternating voltage is used to charge the secondary cell 46 across a charging controller 70, especially a shunt controller. By means of a feedback circuit, the activating or deactivating of the charging function and/or the charge status can be transmitted to the charging module 76, in order to prevent further energy transmission to the module at the end of the charging. At the same time, the feedback serves a protection function, to prevent overheating of foreign bodies, especially metallic ones, that are located above the coil of the charging module. The charge indicator 72 is activated after the inductive energy transmission becomes active and the battery potential is moving in the charging region.

A vibration control, e.g., in the form of a microcontroller and suitable software, with a vibration control memory 58, such as one for the number and duration of the vibrations, can optionally be integrated. In this way, one can create a user profile for evaluation of the therapy outcome. Actuating of the vibration motor 63 then occurs by the vibration control, instead of directly by the comparator 56. The memorized data can be read out serially across an optical interface, i.e., with the help of an optical data transmission unit 60.

FIGS. 10a, 10b and 10c show different views of a charging module 76 or charging station 76. This can be built into a safekeeping container, for example, one in the form of a transport box. The charging module 76 comprises a circuit board 38′ with a transmitter coil 78. This is exposed, i.e., the circuit board 38′ is slotted on the inside, and when installed it is slanted upward about the axis of rotation in order to achieve the closest possible contact with the receiver coil 48 of the vibration module 30 (see FIG. 7a-c). The circuit board 38 is milled in depth on one side in the area of the axis of rotation, so that the electrical connections to a photosensor and to the transmitter coil 78 can only occur on the unmilled side. The end position, i.e., the angle of tilt, can be adapted to the specific installation situation and size of the vibration module 30. The transmitter coil 78 of the charging module 76 is designed in particular as a single-layer flat coil.

FIG. 11 shows a block diagram of a charging module 76. Single arrows here represent a signal, while double arrows represent the energy. Flowing current is represented by V, while the voltage is symbolized by U. Broken lines represent optional components. The signal of a photoreceptor 80 is compared by a comparator 56 against a limit value as the threshold 50′. A monoflop 82, which is retriggerable in particular, is activated. The signal is relayed to a pulse width modulation (PWM) 84, which optionally has a charge monitor 86. Thereupon, a switch 88 is activated, being energized via a wall adapter 90. A charge indicator 72 optionally puts out the charge status. Energy is transmitted via a driver 92, especially a half-bridge, so that a transformer 66, especially a resonance circuit, is provided with energy. The driver 92 receives a signal of an oscillator 94. This oscillator 94, except during a current surge, receives a signal of a comparator 56, which compares the current V against a limit value as the threshold 50′.

The energy transfer from the charging module 76 to the central unit 14 occurs by inductive coupling of two coils in the transmitter and receiver. The transformers 66 in the charging module 76 and in the central unit 14 are operated in resonance. To prevent an unwanted transfer of energy to objects other than the central unit 14 or after the end of the charging, a handshake occurs between the charging module 76 and the central unit 14.

An oscillator 94 generates a cyclical activation signal in the standby mode, regardless of the presence of a receiver, and closes the switch 88 for the energy supply 90 of the charging circuit, e.g., for 100 ms every two seconds, which corresponds to an On time of 5%.

In the central unit 14, a feedback signal is generated upon activating of the internal power supply, especially with the help of a LED. This charge feedback signal is detected in the charging module 76 by means of a photoreceptor 80, especially a photodiode, and taken to a comparator 56. If it exceeds a threshold 50′, the proper power providing of energy to the central unit 14 is recognized and the activation time is increased accordingly, e.g., to an On time of 95%.

In order to further recognize the presence of the central unit 14, a cyclical switching off of the energy transfer occurs in the charging mode, for example, for 100 ms every two seconds, which corresponds to an On time of 95% and an Off time of 5%. If the optical feedback signal of the central unit 14 is still absent during the next activation, the energy transfer is again switched back to the standby mode after a few seconds, with a 5% On time. Also when the end of the charging is reached, the feedback signal is no longer activated, so that no further energy transfer occurs across the inductive interface.

The increasing of the activation time is controlled by a monoflop 82, which must be cyclically reactivated. If the activation pulses are absent, e.g., because the central unit 14 has been removed or the power supply unit 26 is fully charged, the monoflop 82 is no longer retriggered and after its cycle time it switches back to the standby mode.

The control of the On and Off times occurs by means of a PWM stage 84. The control potential is determined by the state of the monoflop 82 and establishes the PWM ratio at 5% or 95%. An adapting to other values is easily possible.

The current V flowing in the coil 78 of the inductive energy transfer is monitored by means of a comparator 56. If it exceeds a threshold 50′, the oscillator 94 is halted and the energy transfer is interrupted. Thus, a safety feature is provided.

The power supply of the charging module 76 comes across a wall adapter 90 or, for example, a USB port of a PC. Optionally, a microcontroller charge monitoring or a voltage feedback of the transmitter coil 78 can be integrated.

In the standard condition, neither the central unit 14 nor the charging module 76 is outfitted with a microcontroller and corresponding software. However, the option exists of outfitting them with a microcontroller, for example for the purpose of collecting user profiles to alter the customer's behavior, to control vibration patterns, or to implement more complex charging methods.

### LIST OF REFERENCE NUMBERS

10 dental arch
12 palate piece
13 base unit
14 central unit
16 pressure sensor, contact sleeve
18 segment
20 conducting material
22 nonconducting material
24, 24′ deep drawn film
26 power supply unit
28 electronics
30 signal generator, vibration module
32 region of the upper incisors
34 region of the lower incisors
36 region of the upper cheek teeth
38, 38′ circuit board
40 motor terminal
42 terminal
44 primary cell
46 secondary cell
48 receiving coil
50, 50′ threshold
52 vibration region
54 filter
56 comparator
58 vibration control memory
60 optical data transmission unit
62 DC/DC converter, dc voltage converter
63 vibration motor
64 display
66 transformer
68 rectifier
70 charging controller
72 charge indicator
73 charge feedback
74 charging, discharging protection
76 charging module, charging station
80 photoreceptor
82 monoflop
84 pulse width modulation (PWM)
86 charge monitor
88 switch
90 wall adapter, power supply
92 driver
94 oscillator
A inner diameter
D thickness
R electrical resistance
t time
V current
U potential

1. Dental splint, comprising
a base unit (13), especially one with dental splint plates irreversibly connected to each other,
at least one power supply unit (26),
at least one signal generator (30), and
at least one pressure sensor (16), which is operatively connected to the power supply unit (26) and the signal generator (30),
wherein the pressure sensor (16) is configured such that it detects both occlusion forces and protrusion forces acting upon it.

2. Dental splint according to claim 1, characterized in that the pressure sensor comprises a contact device (16) with at least one electrical switch contact.

3. Dental splint according to claim 2, characterized in that the contact device comprises at least one contact sleeve (16), which comprises in cross section at least four segments (18) of conducting (20) and nonconducting material (22) alternating in the circumferential direction, especially a plastic material.

4. Dental splint according to claim 3, characterized in that the segments (18) of the contact sleeve (16) essentially extend continuously in the longitudinal direction.

5. Dental splint according to claim 3 characterized in that the contact sleeve (16) is hollow on the inside and/or it comprises at least one elastic and/or flexible material.

6. Dental splint according to claim 3, characterized in that the contact sleeve (16) comprises several sections in the lengthwise dimension, wherein at least one first section, which in the installed condition of the dental splint is arranged especially behind and/or beneath the front incisors, is twisted relative to at least one second section, which in the installed condition of the dental splint is arranged especially at the cuspids, the premolars and/or the molars.

7. Dental splint according to claim 6, characterized in that at least one part of the conducting material (20) in the first section is arranged on either side of a frontal plane and/or at least one part of the conducting material (20) in the second section is arranged on either side of a transverse plane.

8. Dental splint according to claim 1, characterized in that the pressure sensor (16) comprises at least two sensors, which are oriented essentially perpendicular to each other.

9. Dental splint according to claim 1, characterized in that the signal generator (30) comprises a signal transmitter, especially a vibration module, an acoustical signal transmitter, an electrical signal transmitter and/or a thermal signal transmitter.

10. Dental splint according to claim 1, characterized in that it comprises at least one storage, transmitting and/or evaluating unit.

11. Dental splint according to claim 1, characterized in that the signal generator (30) and the power supply unit (26), and also optionally the storage, transmitting and/or evaluating unit, are an integral component of a module, which in particular forms part of the base unit (13), or is itself the base unit (13).

12. Dental splint according to claim 1, characterized in that a central unit (14) which comprises the signal generator (30) and the power supply unit (26), and also optionally the storage, transmitting and/or evaluating unit is at least partially enclosed by a separating film.

13. Dental splint according to claim 1, characterized in that it is formed entirely or partially from at least one thermoplastic polymer and/or at least one elastomer.

14. Dental splint according to claim 1, characterized in that the power supply unit (26) comprises a rechargeable energy store.

15. Charging station (76) for a dental splint according to claim 1, which is designed as a stowage receptacle for the dental splint, wherein the charging station (76) is inductively coupled to the dental splint, so that in particular a rechargeable energy store of the dental splint can be charged.

16. Method for making a dental splint according to claim 1, comprising the providing
of at least one base unit (13), especially one with dental splint plates that are irreversibly connected to each other, being in particular milled or laser cut out from a monolithic block, darned from a fabric, cast from liquid plastic, produced in a polymerization, injection molding, deep drawing or laser sintering process, in the rapid prototyping or 3D printing process, CAD/CAM process or wax process and/or consisting of thermoplastic and/or elastomeric material, especially TPE material,
at least one power supply unit (26), especially one comprising a rechargeable energy store,
at least one signal generator (30) and
at least one pressure sensor (16), which is operatively connected to the power supply unit (16) and the signal generator (30), and
the positioning of the pressure sensor (16) so that it can detect both occlusion forces and protrusion forces acting on it.

17. Method according to claim 16, characterized in that in order to obtain the structure of the pressure sensor (16), a spacer is temporarily introduced into the pressure sensor (16) during the manufacturing of the dental splint.

18. Method for using a dental splint, comprising
the installing of a dental splint, especially according claim 1,
the detecting of the strength of acting occlusion forces and protrusion forces with the help of a pressure sensor (16), especially a contact device fashioned as a contact sleeve, whose electrical resistance (R) changes in dependence on the strength of the occlusion forces and protrusion forces acting upon it,
the relaying of the strength information to a signal generator (30),
the generating of a signal by the signal generator (30) when the strength exceeds a predetermined limit value (50),
the dropping out of the signal when the strength falls below the predetermined limit value (50),
wherein the pressure sensor (16) and the signal generator (30) are energized from a power supply unit (26), comprising in particular a rechargeable energy store.
